(19) United States
(12) Patent Application Publication
(10) Pub. No.: US 2021/0330561 A1
(43) Pub. Date: Oct. 28, 2021

(54) METHOD OF MANUFACTURING DERMATOLOGICALLY ACTIVE SUBSTANCE VEHICLE HAVING MULTILAYER STRUCTURE, AND COMPOSITION MATERIAL FOR FUNCTIONAL COSMETIC CONTAINING DERMATOLOGICALLY ACTIVE SUBSTANCE VEHICLE MANUFACTURED USING THE SAME

(71) Applicants: VENN SKINCARE, INC.; VENN SKINCARE KOREA LLC

(72) Inventor: Jae-Choel MUN, Cheongju-si, Chungcheongbuk-do (KR)

(21) Appl. No.: 16/490,585

(22) PCT Filed: Mar. 7, 2018
(86) PCT No.: PCT/KR2018/002696

§ 371 Date: Sep. 2, 2019

(51) Int. Cl. A61K 8/14; A61K 8/55; A61K 8/92; A61K 8/34; A61Q 19/00

(52) U.S. Cl. CPC A61K 8/14; A61K 8/553; A61Q 19/00; A61K 8/342; A61K 8/92

(57) ABSTRACT

Disclosed is a method of manufacturing a dermatologically active substance vehicle having a multi-layer structure and a composition material for a functional cosmetic containing a dermatologically active substance vehicle manufactured using the same. The method includes manufacturing a hydrogenated lecithin dispersion solution by adding hydrogenated lecithin to purified water at 70 to 90° C. with agitation to thus perform dispersion, manufacturing a pre-emulsion base by adding saturated fatty alcohol having 12 to 22 carbon atoms to the hydrogenated lecithin dispersion solution at 70 to 90° C. with agitation to thus perform dispersion, manufacturing an emulsion base by adding the pre-emulsion base and a water-soluble substance to the purified water at 70 to 90° C. with agitation to thus perform dispersion, and manufacturing a multi-layer globule including phospholipid bilayers by adding oil to the emulsion base at 70 to 90° C. with agitation to thus perform dispersion.

### TECHNICAL FIELD

The present invention relates to a method of manufacturing a dermatologically active substance vehicle having a multi-layer structure and a composition material for a functional cosmetic containing a dermatologically active substance vehicle manufactured using the same. More specifically, the present invention relates to a method of manufacturing a dermatologically active substance vehicle having multiple layers, in which a pre-emulsion base is manufactured using hydrogenated lecithin and saturated fatty alcohol having 12 to 22 carbon atoms and a stable multi-layer globule including phospholipid bilayers is manufactured using the pre-emulsion base to thus be applied to the dermatologically active substance vehicle, and to a composition material for a functional cosmetic containing a dermatologically active substance vehicle manufactured using the same.

### BACKGROUND ART

The skin is broadly divided into three layers, namely the epidermis, dermis, and subcutaneous tissue. The epidermal layer is divided into a stratum corneum, a stratum lucidum, a stratum granulosum, a stratum spinosum, and a stratum basale. Particularly, the stratum corneum is the outermost layer of the skin, which prevents water loss from the skin and protects the skin from external harmful factors.

In the stratum corneum, corneocytes and intercorneocyte lipids form a lamellar layer structure, thus blocking the outflow of water from the body, acting as a defense wall that prevents the entry of harmful substances into the body, acting as a barrier to obstruct the delivery of dermatologically active substances from the stratum corneum of the skin epidermis to the dermis layer, and playing a crucial role in significantly lowering the permeability of dermatologically active substances. Water is contained in an amount of about 10 to 20%. The lamellar layer means that “corneocytes and intercorneocyte lipids are bonded so as to form a bricks-and-mortar structure”. The lamellar layer includes 59% keratin, 31 to 38% natural moisturizing factor (NMF), and 11% intercellular lipids. Particularly, the intercellular lipids include ceramide, cholesterol, and fatty acid.

The stratum granulosum is a layer in which a self-dissolving process is performed. In this layer, the natural moisturizing factor and lipids produced in the stratum spinosum are discharged out and the cell itself is squashed and flattened. In this layer, water content is reduced to remove nuclei and a cornification process actually starts, and the layer blocks 80% of ultraviolet rays. In addition, a protein called filaggrin, known to play an important role in skin barriers, is present in the stratum granulosum, and this protein is known to have a close relationship with hydration of the skin.

The stratum spinosum is the thickest layer in the epidermis, and is a layer which contains a lymph fluid that is involved in the circulation and nutrition supply in the skin and also contains Langerhans cells responsible for skin immunity. The stratum spinosum is a section which mainly synthesizes and maintains necessary ingredients for skin, such as ceramide and moisturizing factors, is a layer in which nutrients move uniformly by exchanging substances between cells, and contains about 70% water.

The stratum basale is a single layer of cuboidal cells which is located at the lowest layer of the epidermis and which is in contact with the dermis, and melanocytes are located therein.

The dermal layer is a layer closely related to the elasticity of the skin, and is about 2 mm thick. This layer includes collagen, which accounts for 90% of the dermis, elastin, which controls the elasticity of the skin, and hyaluronic acid, which controls the water of the skin.

As described above, the skin has both hydrophilic and hydrophobic properties, and the hydrophilic structure and the hydrophobic structure are alternately repeated therein. In particular, the hydrophobic layer exhibits characteristics similar to those of the structure of the cell membrane, and mainly includes ceramide, cholesterol, and fatty acids, which makes it difficult for hydrophilic substances to pass therethrough. With respect thereto, there is a great need to deliver active substances such as nutrients to the dermal layer of the skin using a stable dermatologically active substance vehicle having a multi-layer structure in which hydrophilic and hydrophobic layers alternate with each other. However, to date there has been no research thereon.

With respect to the main channel of the effective ingredient passing through the skin, 1) a method of directly passing the effective ingredient through skin cells, 2) a method of passing the effective ingredient through intercellular spaces in the skin, and 3) a method of passing the effective ingredient through pores are known. Generally, it is thought that the effective ingredient is mainly infiltrated through the pores because the pores are large. However, only about 1% of the effective ingredient infiltrates through the skin itself. In fact, it is known that most effective ingredients infiltrate through the intercellular spaces in the skin, and it can be explained that dermatologically active ingredients are delivered into the skin while the process of partitioning-diffusion is repeated.

In the past, it was thought that only a small-sized liposome of 100 nm or less could pass through the intercellular spaces because the intercellular spaces of the skin are very small, about 30 to 60 nm in size (Journal of Controlled Release 32: 249 (1994)). However, according to a recently published paper (Journal of Controlled Release 59: 87˜97 (1999)), it is difficult for liposomes 100 nm or less in size to actually reach the dermal layer because the liposomes are fused into the cell membrane due to the tension of the cell when passing through the stratum corneum, whereas, on the contrary, largesized liposomes of 500 to 1500 nm can reach the dermal layer. The fundamental principle of infiltration into the skin is not yet fully understood, but liposomes are considered to be able to pass through narrow gaps due to the flexibility of their structure, which resembles water balloons, unlike other micelle structures. However, there is no method of manufacturing a dermatologically active substance vehicle utilizing the stability of the liposome, and therefore there is a great need for development thereof.

Korean Patent No. 10-1057283 discloses a method of manufacturing a water-soluble nano-emulsion containing yolk lecithin, the method including a first step of dissolving lecithin paste and ethanol, a second step of mixing purified water with a lecithin ethanol solution, a third step of performing centrifuging of the mixture, a fourth step of mixing a lower-layer liquid and purified water after the centrifuging, a fifth step of homogenizing the mixture, and a sixth step of filtering a homogeneous suspension. However, in Korean Patent No. 10-1057283, there is still a need to develop a method of manufacturing a stable dermatologically active substance vehicle having a multilayer structure where a hydrophilic layer and a hydrophobic layer alternate with each other using the stability of liposomes.

Four main methods of manufacturing a substance vehicle have been widely used in industry to date, and the methods are as follows.

First (liposome technique): The liposome technique refers to a nano-liposome manufacturing technique, the technique including manufacturing a cosmetic composition material using a general emulsification technique (primary emulsification) and manufacturing liposomes using a micro-fluidizer, which is an expensive apparatus, under specific conditions. However, the liposome technique has problems in that it is difficult to form multiple layers and costs are high due to the use of the micro-fluidizer, which is an expensive apparatus.

Second (multiple emulsification technique): The multiple emulsification technique refers to a manufacturing method using a multiple emulsification technique which includes a process of manufacturing a primary emulsion of a water-in-oil type or an oil-in-water type and manufacturing a secondary emulsion using the primary emulsion as a oil-phase part or a water-phase part (water-in-oil-in-water type (W/O/W type or O/W/O type) emulsion particle structure). However, this technique has problems in that the manufacturing process is complicated and is sensitive to external conditions and in that the formed vehicle is unstable, which makes it difficult to maintain stability against external factors.

Third (encapsulation technique): The encapsulation technique refers to a technique for manufacturing multi-layer emulsion particles using an encapsulation technique that encapsulates and coats a dermatologically active substance using the electrical properties of cations and anions of a polymer substance. However, this technique has problems in that the manufacturing process is complicated and costs are high.

Fourth (liquid crystal emulsification technique): This technique refers to a technique for manufacturing liquid crystal which forms a solid skeletal structure of emulsion particles using the physical and chemical properties of a substance. However, this technique has problems in that it is difficult to form multiple layers, a manufacturing process is complicated, and costs are high.

### DOCUMENTS OF RELATED ART

### Patent Document

(Patent Document 0001) Korean Patent No. 10-1057283

### DISCLOSURE OF INVENTION

### Technical Problem

Accordingly, the present invention has been made keeping in mind the above problems occurring in the prior art, and an object of the present invention is to provide a method of manufacturing a dermatologically active substance vehicle having a multilayer structure. The flexible liposome structure thereof, which resembles water balloons, can smoothly pass through the narrow gaps in the skin, unlike other micelle structures, by manufacturing a stable dermatologically active substance vehicle having a multi-layer structure in which a hydrophilic layer and a hydrophobic layer alternate with each other using a multi-layer globule (liposome structure) including phospholipid bilayers. Therefore, active substances such as nutrients are stably delivered to the dermal layer of the skin.

Another object of the present invention is to provide a method of manufacturing a dermatologically active substance vehicle which has a multi-layer structure and which stably delivers an oil-soluble dermatologically active substance, a water-soluble dermatologically active substance, or a weakly-soluble dermatologically active substance to each layer of the skin while passing through various tissues of the skin, in which a hydrophilic layer and a hydrophobic layer alternate with each other, using the multilayer structure which includes phospholipid bilayers and in which a hydrophilic layer and a hydrophobic layer alternate with each other.

Yet another object of the present invention is to provide a method of easily manufacturing a stable dermatologically active substance vehicle having a multi-layer structure without the need for complicated processes or expensive apparatuses.

Still another object of the present invention is to provide a method of manufacturing a dermatologically active substance vehicle having a multi-layer structure, which is not affected by the surrounding environments such as temperature or humidity, which includes a multi-layer globule including phospholipid bilayers not destroyed despite the passage of time, and in which the multi-layer globule is maintained while passing through various tissues of the skin in which a hydrophilic layer and a hydrophobic layer alternate with each other.

A further object of the present invention is to manufacture various products such as cosmetics using a dermatologically active substance vehicle which has a multi-layer structure and which delivers an oil-soluble dermatologically active substance, a water-soluble dermatologically active substance, or a weakly-soluble dermatologically active substance to each layer of the skin while passing through various tissues of the skin.

### Solution to Problem

In order to accomplish the above objects, the present invention provides a method of manufacturing a dermatologically active substance vehicle having a multi-layer structure. The method includes manufacturing a hydrogenated lecithin dispersion solution by adding hydrogenated lecithin to purified water at 70 to 90° C. with agitation to thus perform dispersion, manufacturing a pre-emulsion base by adding saturated fatty alcohol having 12 to 22 carbon atoms to the hydrogenated lecithin dispersion solution at 70 to 90° C. with agitation to thus perform dispersion, manufacturing an emulsion base by adding the pre-emulsion base and a water-soluble substance to the purified water at 70 to 90° C. with agitation to thus perform dispersion, and manufacturing a multi-layer globule including phospholipid bilayers by adding oil to the emulsion base at 70 to 90° C. with agitation to thus perform dispersion.

In order to accomplish the above objects, the present invention also provides a composition material for a functional cosmetic including a dermatologically active substance vehicle manufactured by the method of manufacturing the dermatologically active substance vehicle.

### Advantageous Effects of Invention

A method of manufacturing a dermatologically active substance vehicle having a multi-layer structure and a composition material for a functional cosmetic containing a dermatologically active substance vehicle manufactured using the same, which are the present invention, have the following effects.

First, there is provided a dermatologically active substance vehicle having a multilayer structure, in which the flexible and stable liposome structure thereof, which resembles water balloons, can smoothly pass through the narrow gaps in the skin, unlike other micelle structures, by manufacturing the dermatologically active substance vehicle having a multi-layer structure in which a hydrophilic layer and a hydrophobic layer alternate with each other using a multi-layer globule (liposome structure) including phospholipid bilayers. Therefore, the dermatologically active substance vehicle stably delivers active substances such as nutrients to the dermal layer of the skin without damaging the vehicle.

Second, the present invention provides a dermatologically active substance vehicle which has a multi-layer structure including phospholipid bilayers in which a hydrophilic layer and a hydrophobic layer alternate with each other. The dermatologically active substance vehicle delivers an oil-soluble dermatologically active substance, a water-soluble dermatologically active substance, or a weakly-soluble dermatologically active substance to each layer of the skin while easily passing through various tissues of the skin in which a hydrophilic layer and a hydrophobic layer alternate with each other.

Third, in the present invention, a stable dermatologically active substance vehicle having a multi-layer structure including at least two phospholipid bilayers may be easily manufactured without the need for complicated processes or expensive apparatuses, whereby economical effects are secured.

Fourth, since the present invention is not affected by the surrounding environments, such as temperature or humidity, and a multi-layer globule including phospholipid bilayers is stably maintained despite the passage of time, an oil-soluble dermatologically active substance, a water-soluble dermatologically active substance, or a weakly-soluble dermatologically active substance is stably delivered to each layer of the skin while passing through various tissues of the skin, in which a hydrophilic layer and a hydrophobic layer alternate with each other.

### BRIEF DESCRIPTION OF DRAWINGS

FIG. 1A is a view showing the reversible micelle structure of a hydrogenated lecithin dispersion solution manufactured by adding hydrogenated lecithin to purified water at 70 to 90° C. with agitation to thus perform dispersion;

FIG. 1B is a view showing the structure of a pre-emulsion base manufactured by adding saturated fatty alcohol having 12 to 22 carbon atoms to the hydrogenated lecithin dispersion solution at 70 to 90° C. with agitation to thus perform dispersion;

FIG. 1C is a view showing the cross-sectional structure of an emulsion base manufactured by adding the pre-emulsion base and a water-soluble substance to the purified water at 70 to 90° C. with agitation to thus perform dispersion;

FIG. 1D is a view showing the cross-sectional structure of a multi-layer globule including phospholipid bilayers manufactured by adding oil to the emulsion base at 70 to 90° C. with agitation to thus perform dispersion;

FIG. 2 is a picture of a dermatologically active substance vehicle having a multilayer structure, which is the present invention, which is taken at a magnification of 400 times using a polarizing microscope;

FIG. 3 is a Raman Spectra comparative chart showing a skin sample (black) which is not treated with a vehicle specimen containing a dermatologically active substance (VENNARC-001) and a skin sample (red) which is treated with a vehicle specimen containing a dermatologically active substance (VENNARC-001);

FIG. 4 is a Raman Spectra comparative chart showing the average of a skin sample (black) which is not treated with a vehicle specimen containing a dermatologically active substance (VENNARC-001) and the average of a skin sample (red) which is treated with a vehicle specimen containing a dermatologically active substance (VENNARC-001);

FIG. 5 is a confocal Raman image showing that a vehicle specimen containing a dermatologically active substance (VENNARC-001) penetrates a skin stratum corneum and infiltrates even into a dermis layer inside an epidermal layer at a peak of 1000 to 1050 cm⁻¹ region (Phenylalanine);

FIG. 6 is a confocal Raman image showing that a vehicle specimen containing a dermatologically active substance (VENNARC-001) penetrates a skin stratum corneum and infiltrates even into a dermis layer inside an epidermal layer at a peak of 2880 to 2935 cm⁻¹ region (CH₂ from skin sample); and

FIG. 7 shows imaging of the infiltration of a vehicle specimen containing a dermatologically active substance (VENNARC-001) into a stratum corneum using the ATR-FTIR imaging study.

### BEST MODE FOR CARRYING OUT THE INVENTION

It is to be understood that the terms or words used in the present specification and claims are not to be construed in a conventional or dictionary sense and that the inventor can properly define the concept of a term to describe their invention in the best possible way. Accordingly, the present invention should be construed as having a meaning and concept consistent with the technical idea of the present invention. Therefore, the embodiments described in the specification of the present invention and the constitutions shown in the drawings are merely the most preferred embodiments of the present invention, and do not represent the entire technical scope of the present invention. It should be understood that various equivalents and modifications that may be substituted for these at the time of filing of the present invention are possible or may be present.

Before describing the present invention with reference to the following Examples, it should be noted that what is not necessary in order to disclose the gist of the present invention, that is, a known constitution that can be obviously added by a person skilled in the art, is not shown in the drawings and not specifically described.

One of the major goals in the cosmetics industry is to find the answer to the question “Is it possible to rejuvenate the skin by delivering dermatologically active substances deep into the skin to slow skin aging and minimize darkness and skin disorders”.

To realize a breakthrough in the answer to this question, the cosmetics industry has been intensively studying various methods of a drug delivery system. A technique for manufacturing water-in-oil-type and oil-in-water-type emulsion particles, which is a conventional cosmetic emulsifying technique, has a merit in that the particles can be easily manufactured by anyone with a little knowledge, but has a big disadvantage in that dermatologically active ingredients are not delivered deep into the skin.

In recent years, nano-liposome, multiple emulsification techniques, encapsulation techniques, and liquid crystal emulsification techniques, which are substance delivery techniques, have improved the performance of delivering a relatively large amount of dermatologically active substances deep into the skin. However, these techniques have drawbacks in that manufacturing is not easy, unlike the conventional emulsification technique, and in that the process is complicated, thus reducing economic feasibility and stability.

Emulsification techniques of various methods are applied to the manufacture of cosmetics, and emulsion particles that are thus manufactured are present in various forms. Among the techniques, recent multi-emulsification techniques, rather than conventional emulsification techniques, have been frequently studied because dermatologically active ingredients or drug ingredients can be effectively delivered to the skin using such multi-emulsification techniques. However, there is a problem in that it is difficult for anyone to easily access these emulsification techniques because special costly apparatuses are required and the manufacturing process is complicated.

Therefore, in the present invention, a typical emulsification method using a system including three phases, namely “water-surfactant-oil”, is used, and a method of easily manufacturing multi-layer vehicles using the apparatus that is conventionally used for the water-in-oil-type emulsification technique or the oil-in-water-type emulsification technique instead of expensive apparatuses is developed.

The emulsification technique for manufacturing the multi-layer vehicle performed in this study includes a process of reducing the high interfacial tension of hydrogenated lecithin, which is used as a surfactant and which has poor compatibility with other substances, using a pre-mixing process so that the hydrogenated lecithin has improved compatibility with other substances (dermatologically active substances). After a pre-emulsion base of the hydrogenated lecithin having improved compatibility with other substances (dermatologically active substance) and saturated fatty alcohol is manufactured, a water-soluble substance is added to form an emulsion base, the emulsion base is added to a cosmetic composition material, and processing conditions such as an agitation time and an agitation speed are set to manufacture a composition material for a functional cosmetic containing a dermatologically active substance vehicle having a multi-layer structure.

In order to improve the skin by applying cosmetics on the skin, a vehicle capable of delivering the dermatologically active ingredients into the skin is required. The surfactant is the most important part in the manufacture of the vehicle. In the present invention, hydrogenated lecithin is used instead of lecithin because of the stability of the vehicle, and the pre-emulsion base is manufactured using hydrogenated lecithin and saturated fatty alcohol having 12 to 22 carbon atoms. As described above, a pretreatment process (pre-mixing) for manufacturing the pre-emulsion base is a key process to lower the high interfacial tension of the hydrogenated lecithin, thus improving compatibility with other substances and also improving the stability of the vehicle. The composition of the pre-emulsion base in this process includes 1) hydrogenated lecithin, 2) saturated fatty alcohol having 12 to 22 carbon atoms, and 3) water as a basic frame, and 1) ceramide, 2) sterol, 3) ethanol, and 4) fatty acid may be added in the manufacture of the pre-emulsion base.

As described above, the pre-treatment process (pre-mixing) for manufacturing the pre-emulsion base in order to lower the high interfacial tension of the hydrogenated lecithin, thus improving the compatibility with other substances and the stability of the vehicle, includes manufacturing the hydrogenated lecithin dispersion solution by adding the hydrogenated lecithin to purified water at 70 to 90° C. with agitation to thus perform dispersion, and manufacturing the pre-emulsion base by adding saturated fatty alcohol having 12 to 22 carbon atoms to the hydrogenated lecithin dispersion solution at 70 to 90° C. with agitation to thus perform dispersion.

Micelles are present in a colloidal dispersion state, and are an aggregate body in which a hydrophilic group portion is directed outwards and a hydrophobic group portion is directed inwards when the concentration of an amphipathic substance reaches or exceeds a predetermined concentration in the case where the amphipathic substance is dissolved in water. The formation of micelles occurs suddenly at a predetermined concentration in the course of dissolution of the amphipathic substance, and the concentration at this point is called a critical micelle concentration. The properties of an aqueous solution are remarkably changed based on the critical micelle concentration. The inside of the micelle is hydrophobic, so oil can be melted therein. The micelles obtained under the conditions of relatively low concentration of typical amphipathic substances form spherical micelles. As the concentration of amphipathic substances increases, the number of spherical micelles gradually increases at an early stage, and finally, the shape of the micelle changes into plates, and micelles having various shapes appear. The factors that determine the critical micelle concentration for forming the micelles having various shapes are temperature, pressure, ionic strength in a solution, and the concentration of an organic substance. One micelle is formed by aggregation of several to several hundred amphipathic substances, and the shape of the micelle includes a sphere, a rod, a plate, or a layer.

When the polarity of the solvent is large, micelles including the hydrophilic groups of amphipathic molecules directed to the outside are formed. When the polarity of the solvent is small, so-called reverse micelles are formed, in which hydrophobic groups are directed outwards. When the solvent affinities of the hydrophilic and hydrophobic groups are almost the same (for example, hydrogenated lecithin), a layered micelle structure is formed.

In this layered structure, a water-soluble substance is contained between sides to which the hydrophilic groups are directed, and a hydrophobic solvent is contained between sides to which the hydrophobic groups are directed. Particularly, a structure present in a state in which a small amount of water is contained in the hydrophilic layer is called a lamella structure, and when a large amount of water is introduced between the hydrophilic groups of the phospholipid lamella, plate-like micelles in which the hydrophilic groups are directed outwards are obtained. A bimolecular membrane, which is a basic membrane structure of the multi-layer globule of the present invention, is obtained by widely developing the plate-like micelles in a transverse direction. Since the end portion (edge) of the layered micelle is unstable due to the contact of the hydrophobic group with water, the layered micelle tends to form a closed structure of a spherical globule containing an aqueous phase therein. Accordingly, a multi-layer structure vehicle forms a spherical shape and becomes stable in an aqueous solution.

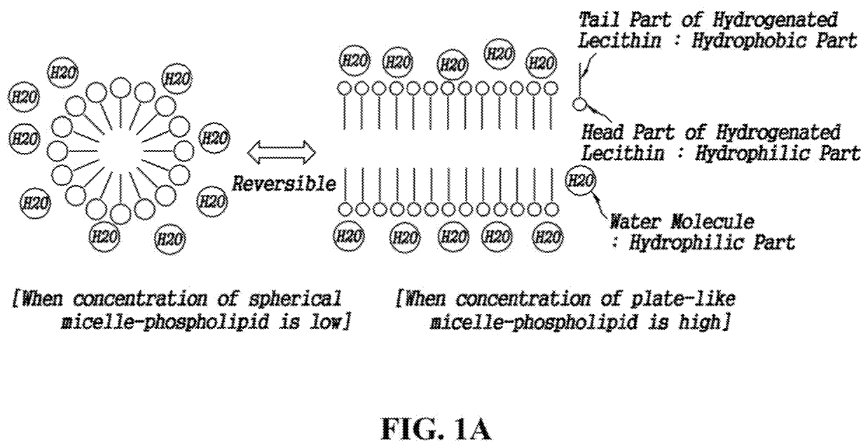

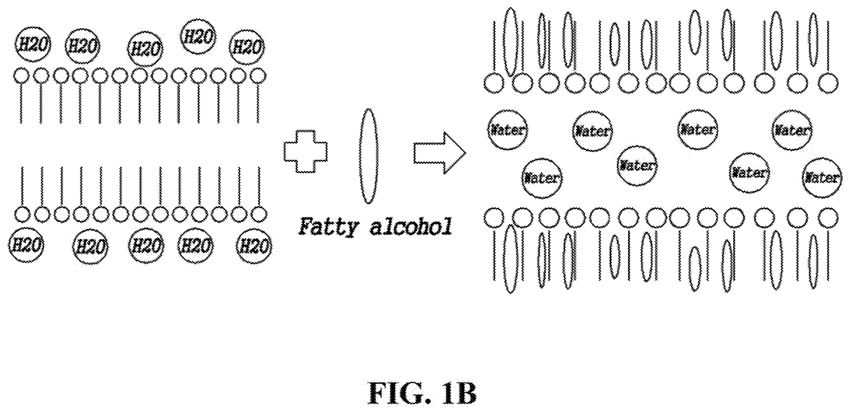

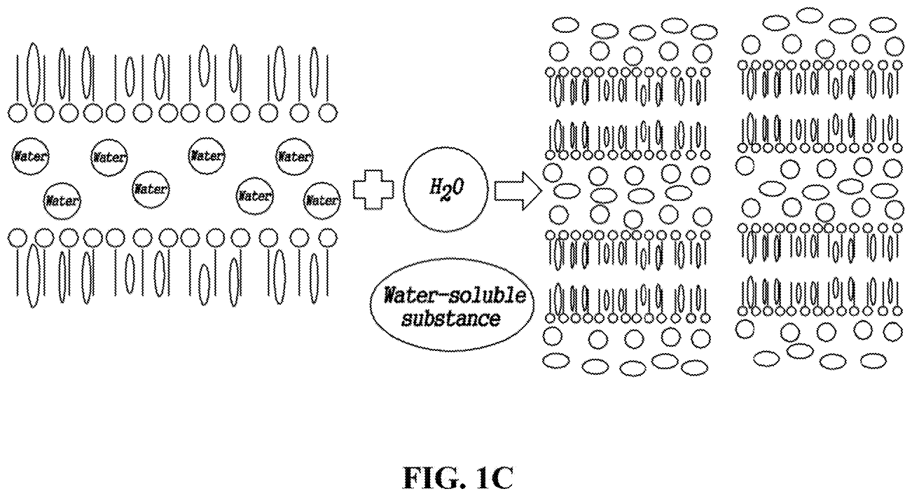

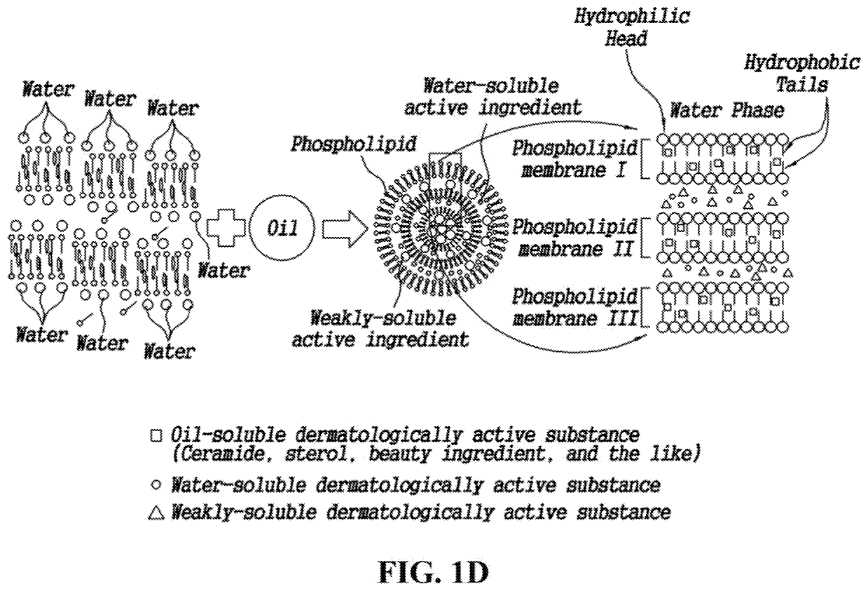

FIG. 1A is a view showing the reversible micelle structure of the hydrogenated lecithin dispersion solution manufactured by adding the hydrogenated lecithin to the purified water at 70 to 90° C. with agitation to thus perform dispersion.

According to FIG. 1A, after the purified water is heated to 70 to 90° C., the hydrogenated lecithin is slowly added thereto with agitation for 8 to 12 minutes (preferably for 10 minutes) under the condition of an AGI mixer (a general mixer) rotation speed of 800 to 1000 rpm, thus manufacturing a hydrogenated lecithin dispersion solution. The manufactured hydrogenated lecithin dispersion solution has a structure in which spherical micelles and plate-like micelles are reversibly present. In the present invention, preferably, the concentration of the phospholipid is controlled to increase so that the plate-like micelles are predominant.

FIG. 1B is a view showing the structure of the pre-emulsion base manufactured by adding the saturated fatty alcohol having 12 to 22 carbon atoms to the hydrogenated lecithin dispersion solution at 70 to 90° C. with agitation to thus perform dispersion.

According to FIG. 1B, the saturated fatty alcohol having 12 to 22 carbon atoms heated to 70 to 90° C. is slowly added to the hydrogenated lecithin dispersion solution with agitation for 8 to 12 minutes (preferably for 10 minutes) under the condition of an AGI mixer (a general mixer) rotation speed of 800 to 1000 rpm, thus manufacturing a pre-emulsion base. The manufactured pre-emulsion base has a structure in which the saturated fatty alcohol is interposed between the hydrogenated lecithins of the plate-like micelles. As described above, the saturated fatty alcohol is interposed between the hydrogenated lecithins of the plate-like micelles, thereby stabilizing the unstable plate-like micelle structure.

FIG. 1C is a view showing the cross-sectional structure of the emulsion base manufactured by adding the pre-emulsion base and the water-soluble substance to the purified water at 70 to 90° C. with agitation to thus perform dispersion.

According to FIG. 1C, the pre-emulsion base and the water-soluble substance heated to 70 to 90° C. are slowly added to the purified water with agitation for 8 to 12 minutes (preferably for 10 minutes) under the condition of an AGI mixer (a general mixer) rotation speed of 800 to 1000 rpm, thus manufacturing an emulsion base. In the manufactured emulsion base, the plate-like micelles are elongated by the water-soluble substance and have a multi-layer structure. Since the plate-like micelles are elongated by the water-soluble substance and have a multi-layer structure, preparation for easy formation of the multi-layer globule is completed. In some cases, a water-soluble dermatologically active substance may be used as the water-soluble substance.

FIG. 1D is a view showing the cross-sectional structure of a multi-layer globule including phospholipid bilayers manufactured by adding oil to the emulsion base at 70 to 90° C. with agitation to thus perform dispersion.

According to FIG. 1D, the oil heated to 70 to 90° C. is slowly added to the emulsion base at 70 to 90° C. with agitation for 8 to 12 minutes (preferably for 10 minutes) under conditions of a homo mixer (a mixer for up-and-down agitation) rotation speed of 3000 to 4000 rpm and a paddle mixer (a mixer for left-and-right agitation) rotation speed of 40 to 60 rpm, thus manufacturing a multi-layer globule including phospholipid bilayers. In the case of the manufactured multi-layer globule including the phospholipid bilayers, the plate-like micelles which are elongated by the water-soluble substance and which have the multi-layer structure wrap the oil and form a closed structure of a spherical globule, and both ends thereof are connected to each other to thus form a spherical vehicle having a multi-layer structure. A plurality of phospholipid bilayers (phospholipid membranes I, II, and III) forms closed structures of a spherical globule. In some cases, an oil-soluble dermatologically active substance may be added to the oil.

The water-soluble substance or the water-soluble dermatologically active substance is located in a water-soluble phase portion between the phospholipid bilayers, and the oil or the oil-soluble dermatologically active substance is located in an oil-soluble phase portion in the phospholipid bilayer. The weakly-soluble dermatologically active substance required in the process of manufacturing cosmetics is located in a water-soluble phase portion between the phospholipid bilayers. Since the dermatologically active substances are separated and are stably present for each layer of the multi-layer structure, the dermatologically active substances such as nutrients may be stably delivered to the dermal layer of the skin without destroying the vehicle.

When the temperature condition and the rotation speed conditions of the mixer, the homo mixer, and the paddle mixer specified in the present invention are not satisfied, the multi-layer globule including the phospholipid bilayers is not manufactured. Particularly, if the process conditions in which the oil heated to 70 to 90° C. is slowly added to the emulsion base at 70 to 90° C. with agitation for 8 to 12 minutes (preferably for 10 minutes) under conditions of a homo mixer (a mixer for up-and-down agitation) rotation speed of 3000 to 4000 rpm and a paddle mixer (a mixer for left-and-right agitation) rotation speed of 40 to 60 rpm are not satisfied, the multi-layer structure may not be formed.

In the step of manufacturing the hydrogenated lecithin dispersion solution, after the hydrogenated lecithin is dissolved in ethanol, the hydrogenated lecithin that is dissolved in ethanol is added to the purified water at 55 to 70° C. with agitation to thus perform dispersion, thereby manufacturing the hydrogenated lecithin dispersion solution. Since the pre-treatment process of ethanol is performed, the temperature of the step of manufacturing the hydrogenated lecithin dispersion solution may be reduced from 70 to 90° C. to 55 to 70° C., thereby preventing the possibility that the hydrogenated lecithin is denatured at high temperatures.

In the step of manufacturing the pre-emulsion base by adding the saturated fatty alcohol having 12 to 22 carbon atoms to the hydrogenated lecithin dispersion solution at 70 to 90° C. with agitation to thus perform dispersion, one or more among ceramide, sterol, or saturated fatty acid having 12 to 22 carbon atoms may be added to the saturated fatty alcohol. In this case, the saturated fatty alcohol having 12 to 22 carbon atoms must be reduced by the amount of the ceramide, the sterol, or the saturated fatty acid having 12 to 22 carbon atoms that is added. However, when the ceramide, the sterol, or the saturated fatty acid having 12 to 22 carbon atoms is added in an excessive amount, the unstable plate-like micelle structure is stabilized, but there is a problem in that the two ends thereof are connected to each other to form a spherical shape before the plate-like micelles form a multi-layer structure.

In the present invention, the pre-emulsion base may be manufactured using 30 to 75 parts by weight of the purified water, 10 to 30 parts by weight of the hydrogenated lecithin, and 15 to 40 parts by weight of the saturated fatty alcohol having 12 to 22 carbon atoms. When the composition of the pre-emulsion base is out of the above-described range, the saturated fatty alcohol is interposed between the hydrogenated lecithins of the plate-like micelles, and thus the effect of stabilizing the unstable plate-like micelle structure may not be obtained. As a result, the dermatologically active substance vehicle having the multi-layer structure may not be manufactured.

In the present invention, the emulsion base may be manufactured using 30 to 75 parts by weight of the purified water, 15 to 40 parts by weight of the pre-emulsion base, and 10 to 30 parts by weight of the water-soluble substance. When the composition of the emulsion base is out of the above-mentioned range, the plate-like micelles which are elongated by the water-soluble substance and which have the multi-layer structure are not formed, so that the multi-layer globule is not easily formed, and as a result, the dermatologically active substance vehicle having the multi-layer structure may not be manufactured.

In the present invention, the multi-layer globule including the phospholipid bilayers may be manufactured using 30 to 75 parts by weight of the emulsion base and 5 to 25 parts by weight of the oil. When the composition of the multi-layer globule including the phospholipid bilayers is out of the above-mentioned range, the plate-like micelles having the multi-layer structure do not wrap the oil and not form a closed structure of a spherical globule. As a result, the spherical dermatologically active substance vehicle having the multi-layer structure may not be manufactured.

In the present invention, 15 to 40 parts by weight of the saturated fatty alcohol having 12 to 22 carbon atoms may include 10 to 30 parts by weight of saturated fatty alcohol having 12 to 20 carbon atoms and 5 to 10 parts by weight of behenyl alcohol. In this case, the reason for adding behenyl alcohol is to reduce the viscosity of the dermatologically active substance vehicle having the multi-layer structure. When the amount of the behenyl alcohol is less than 5 parts by weight, the viscosity reduction effect is very small. When the amount of the behenyl alcohol is more than 10 parts by weight, the viscosity becomes very low.

One or more additives selected from the group consisting of a flavoring agent, a pigment, a stabilizer, a vitamin, a carrier, a bactericide, an antioxidant, a preservative, a moisturizer, a thickener, an inorganic salt, a synthetic polymer substance, oil, water, a surfactant, alcohol, and a chelating agent may be added to the composition material for the functional cosmetic containing the dermatologically active substance vehicle manufactured using the method of manufacturing the dermatologically active substance vehicle, which is the present invention, thus manufacturing functional cosmetics. However, the present invention is not limited to the above-described constitution, and known components used in functional cosmetics may be added.

The functional cosmetic, which is the present invention, may be used for various purposes, such as cosmetic essence, tonic, cosmetic cream, cosmetic lotion, cosmetic beauty pack, cosmetic mist, cosmetic ampoule, cosmetic skin, nutritional cream, skin cream, eye cream, massage cream, cleansing cream, cleansing foam, cleansing water, cleansing oil, milky lotion, soap, liquid cleanser, bathing agent, sunscreen cream, sun oil, shampoo, rinse, hair treatment, hair mousse, hair liquid, pomade, hair-coloring agent, hair-bleaching agent, color rinse, hair tonic, or scalp treatment.

Main Ingredient 1: Hydrogenated Lecithin

Hydrogenated lecithin is a substance in which hydrogen is added to lecithin, which is a diglyceride mixture obtained from beans and eggs, and is a substance having improved stability against oxygen and heat. Lecithin is a kind of phospholipid containing glycerophosphoric acid, and is unstable to oxygen and heat. Lecithin is an important ingredient that decomposes into various fatty acids when hydrolyzed and constitutes cell membranes. This substance has the following characteristics. 1) It helps to form healthy cell membranes as constituents of cells. 2) It keeps water in the skin over a long period of time. 3) It has both hydrophilic and hydrophobic properties, thus acting as an emulsifier in cosmetics. Generally, lecithin is a substance that has a rectangular molecular structure in a plane in which two hydrophobic groups are attached to a hydrophilic portion to thus have both hydrophilic and hydrophobic properties. When dispersed in water, lecithin forms bimolecular membranes instead of spherical micelles. The reason for this is that the two fatty acid chains are so large that they cannot be infiltrated into the micelle, thus forming a bimolecular membrane, that is, a phospholipid bilayer. The resulting bimolecular membrane is a basic factor for forming a vesicle. The closed endoplasmic reticulum is called a vesicle, and among the vesicles, a vesicle including phospholipids is generally called a liposome. The difference between micelles and liposomes is that the micelles are dissolved in water in the form of molecules when diluted but the liposomes are present in a liposome form when diluted. However, since it is difficult to secure the long-term stability of hydrogenated lecithin due to the high interfacial tension thereof, a system capable of lowering the interfacial tension of hydrogenated lecithin is needed. The optimum combination for lowering the high interfacial tension of lecithin and securing long-term stability is a combination of the hydrogenated lecithin and the saturated fatty alcohol having 12 to 22 carbon atoms according to the present invention.

Main Ingredient 2: Usable Saturated Fatty Alcohol

As described above, since it is difficult to secure the long-term stability of hydrogenated lecithin due to the high interfacial tension thereof, a system capable of lowering the interfacial tension of the hydrogenated lecithin is needed. In order to lower the interfacial tension of the hydrogenated lecithin to thus secure the long-term stability thereof, an emulsion stabilizer needs to be used. The emulsion stabilizer means an ingredient that helps form emulsions and stabilize the formed emulsions, and also means a substance that prevents or reduces emulsions from being combined with each other based on mechanisms such as electrical repulsion, change of viscosity, and film formation on emulsion membranes, thus increasing the stability of products. In general, fatty alcohol and fatty acid are representative substances used as emulsion stabilizers in cosmetics. The fatty alcohol plays an important role in enhancing the viscosity of the emulsion and maintaining excellent emulsion stability in the manufacture of emulsions. However, since the fatty acid lowers the viscosity of the emulsion, the fatty acid is unstable when used alone from the aspect of emulsion stability.

In the present invention, the reason why saturated fatty alcohol having 12 to 22 carbon atoms (C12 to C22) is used as the above-described saturated fatty alcohol is that even though the viscosity of the emulsified product is increased when the number of carbon atoms constituting the saturated fatty alcohol is increased, the viscosity is reduced when the number of carbon atoms is 22 or more. Saturated fatty alcohol having 22 carbon atoms may be further mixed to control the viscosity of the emulsified product. Further, saturated fatty alcohol having fewer than 12 carbon atoms does not form a double-membrane (phospholipid bilayer) structure, which is the basic structure of the vehicle, due to the short carbon chain thereof. Even when a double-membrane structure is formed, the stability of the formed vehicle is poor.

In some cases, in order to solve the problem of an increase in the viscosity of the emulsified product as the number of carbon atoms constituting the saturated fatty alcohol increases, a saturated fatty acid having 12 to 22 carbon atoms may be further included. The reason why the added saturated fatty acid has 12 to 22 carbon atoms is that since a saturated fatty alcohol having 12 to 22 carbon atoms is used in the present invention, the compatibility therebetween can be enhanced.

### MODE FOR THE INVENTION

TABLE 1

| | | Classification of | |
|---|---|---|---|
| | Carbon number | fatty acid according | Fatty |
| Fatty acid | & Unsaturation | to carbon number | alcohol |
| Adipic Acid | Carbon number | Short-chain fatty acid | |
| | 6 - diacid | | |
| Suberic Acid | Carbon number | | |
| | 8 - diacid | | |
| Sebacic Acid | Carbon number | | |
| | 10 - diacid | | |
| Lauric | Carbon number | | Lauryl |
| Acid | 12:0 | | Alcohol |
| Myristic | Carbon number | Long-chain fatty acid | Myristyl |
| Acid | 14:0 | | Alcohol |
| Palmitic | Carbon number | | Cetyl |
| Acid | 16:0 | | Alcohol |
| Stearic | Carbon number | | Stearyl |
| Acid | 18:0 | | Alcohol |
| Hydroxystearic | Carbon number | | |
| Acid | 18:0-OH | | |
| Ricinelaidic | Carbon number | | Oleyl |
| Acid | 18:1t-OH | | Alcohol |
| Ricinoleic | Carbon number | | |
| Acid | 18:1c-OH | | |
| Arachidic | Carbon number | | Arachidyl |
| Acid | 20:0 | | Alcohol |
| Behenic | Carbon number | Very-long-chain fatty acid | Behenyl |
| Acid | 22:0 | | Alcohol |

Table 1 shows the types of saturated fatty acid that may be further contained in the saturated fatty alcohol having 12 to 22 carbon atoms (bold letters) usable in the present invention, which are written in bold letters.

### Test Example 1: Pre-Treatment Process for Manufacturing Pre-Emulsion Base

### (Pre-Mixing Process for Manufacturing Multi-Layer Vehicle)

TABLE 2

| Phase | Raw material name | Wt % | Remarks |
|---|---|---|---|
| A | Water | 75.00~30.00 | Solvent |
| B | Hydrogenated | 10.00~30.00 | Emulsifying |
| | lecithin | | agent |
| C | Cetyl alcohol | 10.00~30.00 | Emulsion |
| | orStearyl alcohol or- | | stabilizer |
| | Cetostearyl alcohol | | |
| | or | | |
| D | Behenyl alcohol | 5.00~10.00 | Emulsion |
| | | | stabilizer |
| | Total | 100.00 | |

A pre-emulsion base was prepared in the composition range shown in Table 2 using a pre-treatment process for manufacturing a dermatologically active substance vehicle having a multi-layer structure.

Specific conditions of the pre-treatment process for manufacturing the dermatologically active substance vehicle having the multi-layer structure are as follows.

Process 1: A phase A is put into a main kiln and heated to 80° C.

Process 2: While keeping the temperature at 80° C., a phase B is slowly added to the phase A, agitated, and dissolved (Agitation condition: AGI mixer 900 rpm (or 800 to 1000 rpm), 10 to 20 min).

Process 3: A phase D is put into an auxiliary kiln containing the phase C and is dissolved while the temperature is increased to 80° C. (Agitation condition: AGI mixer 300 rpm).

Process 4: The phase C is put into the phase A at 80° C. and agitated using the AGI mixer to thus perform mixing (Agitation condition: AGI mixer 900 rpm (or 800 to 1000 rpm), 10 to 20 min).

Process 5: The phases A, B, C, and D, mixed by agitation, are cooled to 50° C. and then stored at room temperature in the state of being sealed in a reservoir.

For reference, the constitution of the substance and the pre-treatment process may be modified and changed as described below according to the purpose for use in the skin, such as the maintenance of stability of the dermatologically active substance vehicle having the multi-layer structure, skin protection, and moisturizing. Accordingly, the following additional options are available in the pre-treatment process for manufacturing the pre-emulsion base (pre-mixing process for manufacturing the multi-layer vehicle).

Option 1: As in the step of manufacturing the saturated fatty alcohol dispersion solution of the present invention, a process of firstly dissolving the phase B in ethanol having a purity of 95% or higher and then putting the resultant phase into the phase A may be added when the phase B is put into the phase A and dissolved therein.

Option 2: A ceramide raw material may be added to the phase C in Table 2, thereby performing the pre-treatment process. The saturated fatty alcohol must be removed in an amount that is in proportion to the content of added ceramides when mixing.

Option 3: A sterol raw material may be added in the range of 0.10 to 2.00% to the phase C in Table 2, thereby performing the pre-treatment process. However, the saturated fatty alcohol must be removed in an amount that is in proportion to the content of added sterols when mixing.

Option 4: A predetermined amount of fatty acid may be added to the phase C in Table 2, thereby firmly strengthening the phospholipid membrane including the phospholipid bilayers of the dermatologically active substance vehicle having the multilayer structure or lowering the viscosity of a composition material for the finally manufactured functional cosmetic. The type of fatty acid that can be used is saturated fatty acid having 12 to 22 carbon atoms. Among the acids, long-chain fatty acid and very-long-chain fatty acid are generally used. Among them, behenic acid is particularly preferable. Behenic acid comparatively more strongly affects the viscosity of the composition material for the finally manufactured functional cosmetic than other long-chain fatty acids or very-long-chain fatty acids. Accordingly, the content thereof must be carefully controlled. However, the saturated fatty alcohol must be removed in an amount that is in proportion to the content of added fatty acid when mixing.

In Table 2, the constitution of the substance includes 1) purified water, 2) hydrogenated lecithin, and 3) saturated fatty alcohol. The saturated fatty alcohol acts as an emulsion stabilizer and is used in order to lower the high interfacial tension of the hydrogenated lecithin, and the hydrogenated lecithin having both hydrophilic and hydrophobic properties acts as an emulsifier. In addition, the saturated fatty alcohol that is used may be used alone or in a combination of two or more thereof, and saturated fatty alcohol having 12 to 22 carbon atoms is used as a main ingredient. In order to lower the viscosity of a product obtained by emulsification, behenyl alcohol having 22 carbon atoms may be further used in combination therewith.

In order to positively affect the physical properties of the dermatologically active substance vehicle, the state of emulsion particles, and the manufacturing process when the composition material for the pre-emulsion base is prepared, 1) ceramide, 2) sterol, 3) fatty acid, and 4) ethanol may be added to modify the composition material. For example, in order to strengthen the phospholipid bilayers (phospholipid membranes I, II, and III) of the multi-layer structure emulsion particle, one or more of ceramide, sterol, or saturated fatty acid having 12 to 22 carbon atoms may be added during the step of manufacturing the pre-emulsion base by adding the saturated fatty alcohol having 12 to 22 carbon atoms to the hydrogenated lecithin dispersion solution at 70 to 90° C. with agitation to thus perform dispersion. Since the hydrogenated lecithin has a hydrophilic head and a hydrophobic tail and is not high in terms of solubility in water at room temperature, after the hydrogenated lecithin is first dissolved in ethanol, the hydrogenated lecithin that is dissolved in ethanol may be added to purified water heated to 55 to 70° C. with agitation to thus perform dispersion.

Further, care should be taken to control the temperature in the pre-treatment process (pre-mixing) for manufacturing the dermatologically active substance vehicle having the multi-layer structure. When the temperature is maintained at 70 to 90° C. over a long period of time, the hydrogenated lecithin is hardened due to the temperature effect, and the function of the hydrogenated lecithin is remarkably weakened. As a result, the ability to generate a vesicle, which is a closed endoplasmic reticulum, that is, a liposome, is lowered, and the stability and usability of the composition material for the functional cosmetic and the functional cosmetic are deteriorated due to the decrease in the stability of the dermatologically active substance vehicle that is generated. Therefore, it is recommended to cool the pre-emulsion base produced using the pretreatment process (pre-mixing) for manufacturing the dermatologically active substance vehicle as fast as possible and to store the cooled pre-emulsion base at room temperature. Accordingly, in the case where a step of manufacturing an emulsion base by adding the pre-emulsion base and the water-soluble substance to the purified water at 70 to 90° C. with agitation to thus perform dispersion and a step of manufacturing a multi-layer globule including phospholipid bilayers by adding oil to the emulsion base at 70 to 90° C. with agitation to thus perform dispersion are not successively performed immediately after the step of manufacturing the pre-emulsion base by adding the saturated fatty alcohol having 12 to 22 carbon atoms to the hydrogenated lecithin dispersion solution at 70 to 90° C. with agitation to thus perform dispersion, it is preferable to further include a step of cooling the pre-emulsion base at room temperature between the step of manufacturing the pre-emulsion base and the step of manufacturing the emulsion base in order to prevent the hardening of the hydrogenated lecithin and the weakening of the ability to generate liposomes.

In addition, the hydrogenated lecithin has a hydrophilic head and a hydrophobic tail, and is not high in terms of solubility in water at room temperature. In this regard, there are 1) a method of manufacturing a pre-emulsion base by adding saturated fatty alcohol having 12 to 22 carbon atoms to a hydrogenated lecithin dispersion solution at 70 to 90° C. with agitation to thus perform dispersion, and 2) a method of dissolving hydrogenated lecithin in ethanol and then adding the hydrogenated lecithin dissolved in ethanol to purified water heated to 55 to 70° C. with agitation to thus perform dispersion. As a result, it is possible to disperse the hydrogenated lecithin in water using the above two methods, and there is no difference in the degree of dispersion or physical properties of the two methods regardless of which method is selected because the selection relates to shortening of the dispersion process time and the efficiency.

Ceramide and sterol are a kind of lipid membrane ingredients of the epidermal stratum corneum among skin cells. They are substances that prevent water loss from the skin surface and block the infiltration of harmful substances from the outside. Ceramide plays a role as a barrier to lipid layers by structurally bonding with water, and has functions of protecting the body from the external environment and microorganisms and of regulating cell growth as a signaling system in terms of physiology. The role of ceramide and sterol in the process of manufacturing the dermatologically active substance vehicle having the multi-layer structure is preferably to arrange hydrogenated lecithin particles and to be provided between the hydrogenated lecithin particles having a double-membrane structure including the phospholipid bilayers so that the membrane of the double-membrane structure is made more dense, thus improving the stability of the dermatologically active substance vehicle having the multi-layer structure. As shown in Table 2, finally, the pre-emulsion base obtained during the pre-treatment process for manufacturing the dermatologically active substance vehicle having the multi-layer structure is preferably used in a content in the range of 10.00 to 20.00 wt % in the composition material for the functional cosmetic containing the dermatologically active substance vehicle.

2. Test Example 2: Manufacture of composition material for functional cosmetic containing dermatologically active substance vehicle having multi-layer structure using pre-emulsion base

TABLE 3

| Phase | Raw material name | Content | Remarks |
|---|---|---|---|
| A | Purified water | 47.45 | |
| | Glycerin, Panthenol | 2.00 | |
| | Pre-Mixing Base composition material | 16.00 | |
| | Disodium EDTA, Allantoin, L-Arginine | 0.30 | |
| | Nylon-12, Xanthan Gum | 1.05 | |
| | VENNARC-001 Bioactives | 14.00 | |
| | Sodium Olivoil Glutamate | 1.00 | |
| B | Kalcohol 6870P | 1.50 | |
| | Stearic Acid | 0.50 | |
| | Sunflower Oil, Hazelnut Oil, Coco- | 9.70 | |
| | glycerides, Bees Wax, Shea Butter | | |
| | Glyceryl Stearate | 1.50 | |
| C | Carbopol 940(2%) | 5.00 | |
| | Total | 100.00 | |

Table 3 shows the composition of a composition material for a functional cosmetic containing a vehicle containing a dermatologically active substance (VENNARC-001 BIOACTIVES).

A method of manufacturing the composition material for the functional cosmetic containing the vehicle containing the dermatologically active substance (VENNARC-001 BIOACTIVES) as shown in Table 3 is as follows.

Process 1: A phase A is put into a main kiln and then heated to 80° C. with agitation to thus perform dissolving. (Agitation condition: Paddle mixer 50 rpm)

Process 2: The phase A is dissolved while being heated to 80° C., and then uniformly dispersed using a paddle mixer and a homo mixer (Agitation conditions: Paddle mixer 50 rpm, Homo mixer 3000 rpm, 10 min at 80° C.).

Process 3: A phase B is put into an auxiliary kiln and then dissolved while being heated to 80° C. (Agitation condition: Dispersing mixer 200 rpm).

Process 4: The phase B is put into the phase A, and then emulsified with agitation under a constant temperature condition of 80° C. (Agitation conditions: Paddle mixer 50 rpm, Homo mixer 3000 rpm, 8 min, at 80° C.).

Process 5: The contents contained in the main kiln are cooled to 45° C. with deaeration (Agitation conditions: Paddle mixer 40 rpm, Cooling up to 40° C. under vacuum).

Process 6: The phase C is put into the main kiln and the contents are then uniformly agitated (Agitation conditions: Paddle mixer 40 rpm, Homo mixer 2200 rpm, 4 min).

Process 7: The contents contained in the main kiln are subjected to second deaeration while being cooled to 35° C. (Agitation conditions: Paddle mixer 40 rpm, Cooling up to 35° C. under vacuum).

Process 8: The contents contained in the main kiln are floated in a separate reservoir and then aged at room temperature for 3 days.

Particularly, when in process 2, uniform dispersion is not performed at a temperature of 80° C. for 10 min under conditions of a homo mixer (a mixer for up-and-down agitation) rotation speed of 3000 rpm and a paddle mixer (a mixer for left-and-right agitation) rotation speed of 50 rpm, or when in process 4, uniform dispersion is not performed at a temperature of 80° C. for 8 min under conditions of a homo mixer (a mixer for up-and-down agitation) rotation speed of 3000 rpm and a paddle mixer (a mixer for left-and-right agitation) rotation speed of 50 rpm, the dermatologically active substance vehicle having the multi-layer globule and the multi-layer structure including the phospholipid bilayers may not be formed.

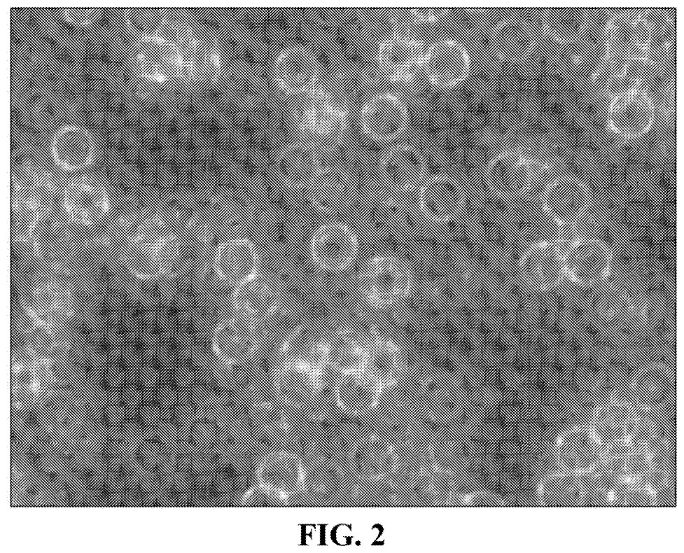

FIG. 2 is a picture of a dermatologically active substance vehicle having a multilayer structure, which is the present invention, taken at a magnification of 400 times using a polarizing microscope.

From FIG. 2, it could be confirmed that the vehicle specimen containing the dermatologically active substance (VENNARC-001) had the multi-layer structure including a plurality of phospholipid bilayers in the composition material for the functional cosmetic.

3. Example 1: Manufacture of cosmetic lotion that contains composition material for functional cosmetic containing dermatologically active substance vehicle having multilayer structure (multi-layer vehicle) using pre-emulsion base obtained via pretreatment process

TABLE 4

| Phase | Raw material name | Content | Remarks |
|---|---|---|---|
| A | Purified water | 61.71 | |
| | Glycerin | 5.00 | |
| | Pre-Mixing Base | 10.00 | |
| | Disodium EDTA, Allantoin, L-Arginine | 0.24 | |
| | Xanthan Gum | 0.05 | |
| | VENNARC-001 Bioactives | 5.00 | |
| B | Kalcohol 6870P | 1.00 | |
| | Stearic Acid | 0.50 | |
| | Vegetable Oil | 8.00 | |
| | Bees Wax | 0.50 | |
| | Glyceryl Stearate | 1.00 | |
| C | Carbomer | 5.00 | |
| E | 1,2-Hexanediol | 2.00 | |
| | Total | 100.00 | |

4. Example 2: Manufacture of cosmetic essence that contains composition material for functional cosmetic containing dermatologically active substance vehicle having multi-layer structure (multi-layer vehicle) using pre-emulsion base obtained via pretreatment process

TABLE 5

| Phase | Raw material name | Content | Remarks |
|---|---|---|---|
| A | Purified water | 58.43 | |
| | Glycerin | 5.00 | |
| | Pre-Mixing Base | 12.00 | |
| | Disodium EDTA, Allantoin, L-Arginine | 0.35 | |
| | Xanthan Gum | 0.08 | |
| | VENNARC-001 Bioactives | 8.00 | |
| B | Kalcohol 6870P | 1.50 | |
| | Stearic Acid | 0.50 | |
| | Vegetable Oil | 8.00 | |
| | Bees Wax | 0.80 | |
| | Glyceryl Stearate | 1.20 | |
| C | Carbomer | 0.14 | |
| D | 1,2-Hexanediol | 2.00 | |
| E | Beta-Glucan | 2.00 | |
| | Total | 100.00 | |

5. Example 3: Manufacture of skin cream that contains composition material for functional cosmetic containing dermatologically active substance vehicle having multilayer structure (multi-layer vehicle) using pre-emulsion base obtained via pretreatment process

TABLE 6

| Phase | Raw material name | Content | Remarks |
|---|---|---|---|
| A | Purified water | 40.68 | |
| | Glycerin | 10.00 | |
| | Pre-Mixing Base | 18.00 | |
| | Disodium EDTA, Allantoin, L-Arginine | 0.45 | |
| | Xanthan Gum | 0.06 | |
| | VENNARC-001 Bioactives | 10.00 | |
| | Ceramide NP | 0.50 | |
| | Sodium Hyaluronate(1%) | 1.00 | |
| B | Cetostearyl Alcohol | 1.80 | |
| | Stearic Acid | 0.80 | |
| | Sunflower Oil | 10.0 | |
| | Bees Wax | 0.80 | |
| | Glyceryl Stearate | 1.60 | |
| C | Carbomer | 0.16 | |
| D | 1,2-Hexanediol | 2.00 | |
| | Beta-Glucan | 2.00 | |
| | Pycnogenol | 0.05 | |
| | Green Tea Catechins | 0.10 | |
| | Total | 100.00 | |

6. Example 4: Manufacture of eye cream that contains composition material for functional cosmetic containing dermatologically active substance vehicle having multilayer structure (multi-layer vehicle) using pre-emulsion base obtained via pretreatment process

TABLE 7

| Phase | Raw material name | Content | Remarks |
|---|---|---|---|
| A | Purified water | 48.85 | |
| | Glycerin | 5.00 | |
| | Pre-Mixing Base | 16.00 | |
| | Disodium EDTA, Allantoin, L-Arginine | 0.33 | |
| | Xanthan Gum | 0.08 | |
| | VENNARC-001 Bioactives | 10.00 | |
| | Ceramide NP | 0.50 | |
| | Beta-Glucan | 3.00 | |
| B | Cetostearyl Alcohol | 1.50 | |
| | Stearic Acid | 0.50 | |
| | Bees Wax | 0.60 | |
| | Hazel Nut Oil | 8.00 | |
| | Phytosterol | 1.00 | |
| | Glyceryl Stearate | 1.50 | |
| | Arlacel 165 | 1.00 | |
| C | Carbomer | 0.14 | |
| D | 1,2-Hexanediol | 2.00 | |
| | Total | 100.00 | |

Confocal Raman spectroscopy was used in order to confirm whether the vehicle specimen containing the dermatologically active substance having the multi-layer structure according to the present invention (VENNARC-001) was infiltrated into the skin (Investigation of formula delivery via confocal Raman spectroscopy).

\textcircled{1} Object: In-Vitro Skin Sample Test (Ex-Vivo Skin Test)

In order to study and evaluate the delivery of the vehicle specimen containing the dermatologically active substance (VENNARC-001) to ex-vivo skin samples, accumulation and infiltration of the vehicle specimen containing the dermatologically active substance (VENNARC-001) were analyzed using confocal Raman spectroscopy.

\textcircled{1} Measurement (Confocal Raman Study)

Confocal Raman spectroscopy was used to study the infiltration and accumulation of the vehicle specimen containing the dermatologically active substance (VENNARC-001) in the stratum corneum and the epidermal layer. As the substrate, an ex-vivo human skin sample was purchased from a contracted provider, and both a skin control sample (Sample 1) on which no action was taken and a skin sample (Sample 2) which was treated with the vehicle specimen containing the dermatologically active substance having the multi-layer structure (VENNARC-001) were prepared. The skin was treated according to an experimental protocol using the skin sample (Sample 2) which was treated with the vehicle specimen containing the dermatologically active substance having the multi-layer structure (VENNARC-001). (Experimental protocol: the vehicle specimen containing the dermatologically active substance having the multi-layer structure (VENNARC-001) was applied on the skin and the skin is massaged for 30 seconds. In addition, the vehicle specimen containing the dermatologically active substance (VENNARC-001) was kept on the surface of the skin for 30 min for the purpose of transdermal absorption (Franz cell) before measurement.)

\textcircled{3} Measurement Range (Areas for Measurements)

Measurement using confocal Raman spectroscopy was performed at the following points over the skin sample (Sample 1) which was not treated with the vehicle specimen containing the dermatologically active substance (VENNARC-001) and the skin sample (Sample 2) which was treated with the vehicle specimen containing the dermatologically active substance (VENNARC-001).

1) Four points on the surface of the skin (X-axis—scans, measured at 5 an intervals)

2) Depth 6 points—The surface of the skin and five points beneath the surface (Z-axis—scans, measured at every 5 μm intervals).

Skin samples were analyzed using confocal Raman spectroscopy under the following analysis conditions (Analysis conditions: Laser excitation 532 nm/Exposition time 30 seconds/Spectral range 4000-400 cm⁻¹/10 Scans accumulation).

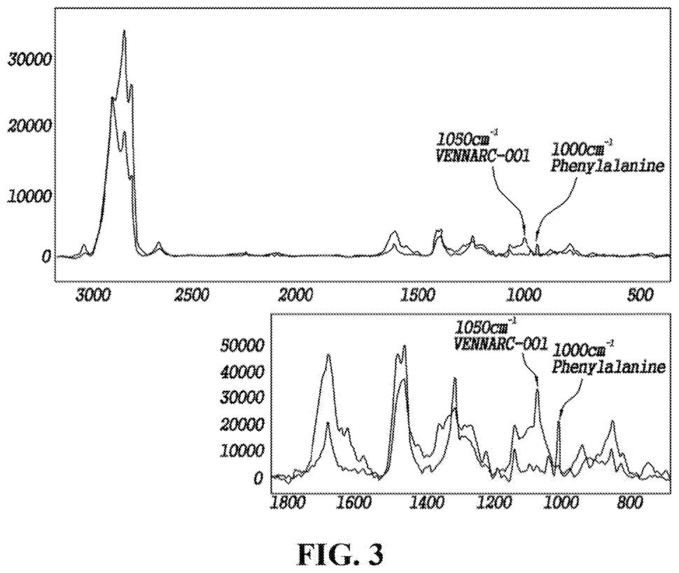

FIG. 3 is a Raman Spectra comparative chart showing the skin sample (sample 1, black) which was not treated with the vehicle specimen containing the dermatologically active substance (VENNARC-001) and the skin sample (sample 2, red) which was treated with the vehicle specimen containing the dermatologically active substance (VENNARC-001).

According to FIG. 3, an optimal marker for confirming the vehicle specimen containing the dermatologically active substance (VENNARC-001) in confocal Raman spectroscopy is a non-overlapping peak of 1050 cm⁻¹ in a Raman skin contribution. A strong IR peak around 2880 cm⁻¹ may be used as another marker of the vehicle specimen containing the dermatologically active substance (VENNARC-001). Therefore, it can be confirmed that the best marker is 1050 or 2880 cm⁻¹ in tracking the delivery of the vehicle specimen containing the dermatologically active substance (VENNARC-001) into the skin.

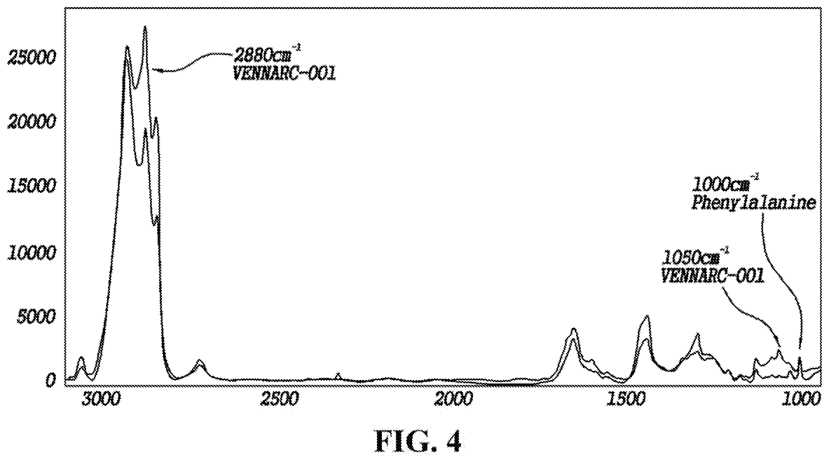

FIG. 4 is a Raman Spectra comparative chart showing the average of the skin sample (black) which is not treated with the vehicle specimen containing the dermatologically active substance (VENNARC-001) and the average of the skin sample (red) which is treated with the vehicle specimen containing the dermatologically active substance (VENNARC-001).

From FIG. 4, it could be confirmed that the best marker was 1050 or 2880 cm⁻¹ in tracking the delivery of the vehicle specimen containing the dermatologically active substance (VENNARC-001) into the skin.

A special confocal Raman image was formed in order to confirm whether the vehicle specimen containing the dermatologically active substance (VENNARC-001) is infiltrated into the treated skin sample (Sample 2) using imaging. In the confocal Raman image below, an increase in the extent of the red color shows deeper infiltration and infiltration of more vehicle specimens containing the dermatologically active substance (VENNARC-001) into the skin.

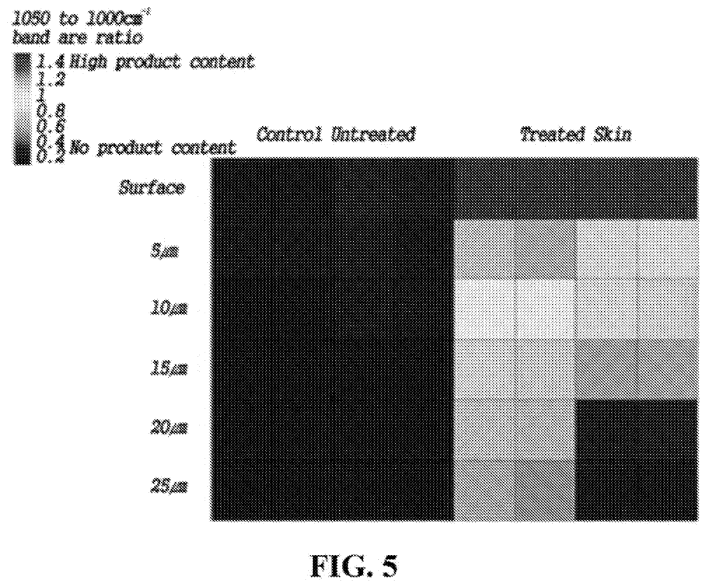

FIG. 5 is a confocal Raman image showing that the vehicle specimen containing the dermatologically active substance (VENNARC-001) penetrates a skin stratum corneum and infiltrates even into a dermis layer inside an epidermal layer at a peak of 1000 to 1050 cm⁻¹ region (Phenylalanine).

In the skin sample (Sample 2) treated with the vehicle specimen containing the dermatologically active substance (VENNARC-001) as shown in the confocal Raman image, it could be seen that the vehicle specimen containing the dermatologically active substance (VENNARC-001) penetrated the stratum corneum in the skin and further infiltrated into the inside of the epidermal layer. In a specific region, it could be confirmed that the vehicle specimen containing the dermatologically active substance (VENNARC-001) was infiltrated into even below the range of measurement performed in this study. As expected, nothing was detected in the skin sample (Sample 1) that was not treated with the vehicle specimen containing the dermatologically active substance (VENNARC-001).

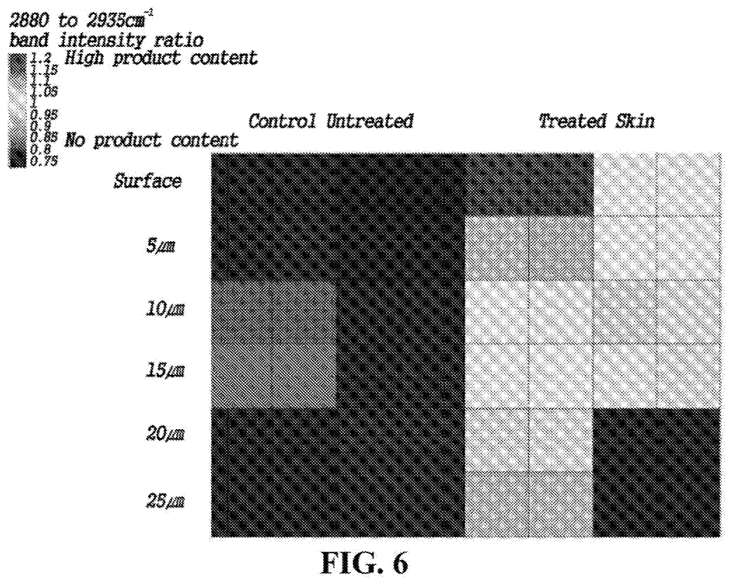

FIG. 6 is a confocal Raman image showing that the vehicle specimen (VENNARC-001) containing the dermatologically active substance penetrates the skin stratum corneum and infiltrates even into a dermis layer inside an epidermal layer at a peak of 2880 to 2935 cm⁻¹ region (CH₂ from skin sample).

In order to further confirm this result, measurement was performed using another marker (a strong IR peak around 2880 cm⁻¹) of the vehicle specimen containing the dermatologically active substance (VENNARC-001), and like the preceding, it could be confirmed that the vehicle specimen containing the dermatologically active substance (VENNARC-001) penetrated the stratum corneum in the skin and further infiltrated into the inside of the epidermal layer in the skin sample (Sample 2) treated with the vehicle specimen containing the dermatologically active substance (VENNARC-001).

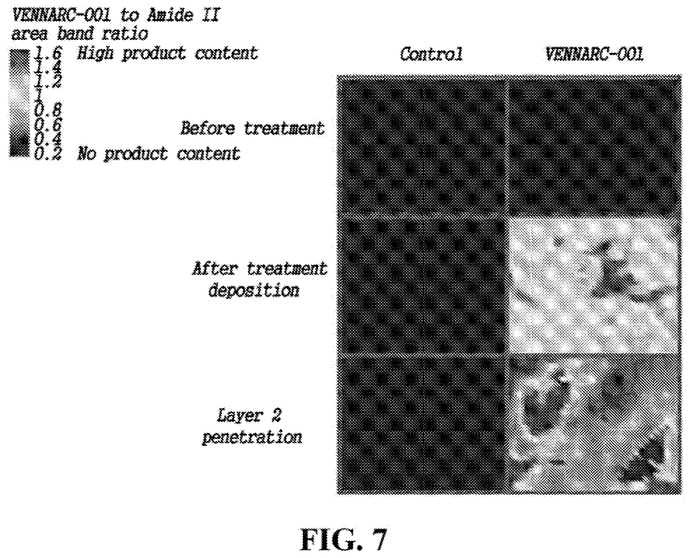

FIG. 7 shows imaging of the infiltration of the vehicle specimen containing the dermatologically active substance (VENNARC-001) into the stratum corneum using the ATR-FTIR imaging study.

According to FIG. 7, the infiltration of the vehicle specimen containing the dermatologically active substance (VENNARC-001) into the stratum corneum could be confirmed using the ATR-FTIR imaging study. From the comparison of the skin sample (Sample 1, Control), which was not treated with the vehicle specimen containing the dermatologically active substance (VENNARC-001), to the skin sample (Sample 2, VENNARC-001), which was treated with the vehicle specimen containing the dermatologically active substance (VENNARC-001), it can be seen that the skin sample (Sample 1, Control) not treated with the vehicle specimen containing the dermatologically active substance (VENNARC-001) exhibited a blue color before and after the specimen treatment, showing no infiltration of the vehicle specimen containing the dermatologically active substance (VENNARC-001) into the stratum corneum. Conversely, it could be confirmed that the skin sample (Sample 2, VENNARC-001) treated with the vehicle specimen containing the dermatologically active substance (VENNARC-001) exhibited a blue color before the specimen treatment but exhibited the increased red color after the specimen treatment, showing active infiltration of the vehicle specimen containing the dermatologically active substance (VENNARC-001) into the stratum corneum. As shown in FIG. 7, an increase in the red color shows the increased amount of the vehicle specimen containing the dermatologically active substance (VENNARC-001) delivered into the stratum corneum, whereby the effect of the present invention was confirmed.

While the present invention has been described with reference to exemplary embodiments thereof, it is to be understood that the present invention is not limited to the disclosed exemplary embodiments, but on the contrary, those skilled in the art will appreciate that various amendments and modifications are possible from the description. Accordingly, it is intended that the idea of the present invention be defined only by the claims appended hereto, and that all equivalents or equivalent variations thereof fall within the scope of the present invention.

1. A method of manufacturing a dermatologically active substance vehicle having a multi-layer structure, the method comprising:
manufacturing a hydrogenated lecithin dispersion solution by adding hydrogenated lecithin to purified water at 70 to 90° C. with agitation to thus perform dispersion;
manufacturing a pre-emulsion base by adding saturated fatty alcohol having 12 to 22 carbon atoms to the hydrogenated lecithin dispersion solution at 70 to 90° C. with agitation to thus perform dispersion;
manufacturing an emulsion base by adding the pre-emulsion base and a water-soluble substance to the purified water at 70 to 90° C. with agitation to thus perform dispersion; and
manufacturing a multi-layer globule including phospholipid bilayers by adding oil to the emulsion base at 70 to 90° C. with agitation to thus perform dispersion.

2. The method of claim 1,
wherein the manufacturing the multi-layer globule including the phospholipid bilayers is performed for 8 to 12 minutes under conditions of a homo mixer rotation speed of 3000 to 4000 rpm and a paddle mixer rotation speed of 40 to 60 rpm.

3. The method of claim 1,
wherein the manufacturing the hydrogenated lecithin dispersion solution is performed for 8 to 12 minutes under a condition of a mixer rotation speed of 800 to 1000 rpm, the manufacturing the pre-emulsion base is performed for 8 to 12 minutes under a condition of a mixer rotation speed of 800 to 1000 rpm, and the manufacturing a composition material base for skin moisturizing is performed for 8 to 12 minutes under a condition of a mixer rotation speed of 800 to 1000 rpm.

4. The method of claim 1, wherein the manufacturing the hydrogenated lecithin dispersion solution includes dissolving the hydrogenated lecithin in ethanol and then adding the hydrogenated lecithin dissolved in the ethanol to the purified water at 55 to 70° C. with agitation to thus perform dispersion, thereby manufacturing the hydrogenated lecithin dispersion solution.

5. The method claim 1, wherein one or more among ceramide, sterol, or saturated fatty acid having 12 to 22 carbon atoms are added to the saturated fatty alcohol.

6. The method claim 1, wherein the water-soluble substance is a water-soluble dermatologically active substance, and an oil-soluble dermatologically active substance is added to the oil.

7. The method of claim 6, wherein the water-soluble dermatologically active substance is located between phospholipid bilayers and the oil-soluble dermatologically active substance is located in the phospholipid bilayers.

8. The method of claim 1, wherein the pre-emulsion base is manufactured using 30 to 75 parts by weight of the purified water, 10 to 30 parts by weight of the hydrogenated lecithin, and 15 to 40 parts by weight of the saturated fatty alcohol having 12 to 22 carbon atoms, the emulsion base is manufactured using 30 to 75 parts by weight of the purified water, 15 to 40 parts by weight of the pre-emulsion base, and 10 to 30 parts by weight of the water-soluble substance, and the multilayer globule including the phospholipid bilayers is manufactured using 30 to 75 parts by weight of the emulsion base and 5 to 25 parts by weight of the oil.

9. The method of claim 8, wherein 15 to 40 parts by weight of the saturated fatty alcohol having 12 to 22 carbon atoms includes 10 to 30 parts by weight of the saturated fatty alcohol having 12 to 20 carbon atoms and 5 to 10 parts by weight of behenyl alcohol.

10. A composition material for a functional cosmetic comprising:
a dermatologically active substance vehicle manufactured by the method of manufacturing the dermatologically active substance vehicle of claim 1.

11. The composition material of claim 10, further comprising:
one or more additives selected from the group consisting of a flavoring agent, a pigment, a stabilizer, a vitamin, a carrier, a bactericide, an antioxidant, a preservative, a moisturizer, a thickener, an inorganic salt, a synthetic polymer substance, oil, water, a surfactant, alcohol, and a chelating agent.

12. The composition material of claim 11,
wherein the composition material is cosmetic essence, tonic, cosmetic cream, cosmetic lotion, cosmetic beauty pack, cosmetic mist, cosmetic ampoule, cosmetic skin, nutritional cream, massage cream, cleansing cream, cleansing foam, cleansing water, cleansing oil, milky lotion, soap, liquid cleanser, bathing agent, sunscreen cream, sun oil, shampoo, rinse, hair treatment, hair mousse, hair liquid, pomade, hair-coloring agent, hair-bleaching agent, color rinse, hair tonic, or scalp treatment.
